(19) United States
(12) Patent Application Publication
(10) Pub. No.: US 2023/0000475 A1
(43) Pub. Date: Jan. 5, 2023

(54) BIOLOGICAL SAMPLING DEVICE AND METHOD OF USE

(71) Applicant: Case Western Reserve University, Cleveland, OH (US)

(72) Inventors: Sanford Markowitz, Cleveland, OH (US); Amitabh Chak, Cleveland, OH (US); Joseph Willis, Cleveland, OH (US); Lishan Aklog, Purchase, NY (US); Richard Yazbeck, Norwell, MA (US); Michael Boutillette, San Francisco, CA (US); Brian deGuzman, Paradise Valley, AZ (US); David Wurtman, New York, NY (US)

(73) Assignee: Case Western Reserve University, Cleveland, OH (US)

(21) Appl. No.: 17/855,530

(22) Filed: Jun. 30, 2022

Related U.S. Application Data
(60) Provisional application No. 63/217,823, filed on Jul. 2, 2021.

(51) Int. Cl. A61B 10/04

(52) U.S. Cl. CPC A61B 10/04; A61B 2010/0216

(57) ABSTRACT

Provided herein are methods and a device for collecting biological samples. In particular, the present disclosure relates to a device designed to specifically capture samples within the target areas of a patient for specific cell collection for particular diagnosis.

### RELATED APPLICATION

This application claims priority to and the benefit of U.S. Provisional Patent Application No. 63/217,823, filed Jul. 2, 2021, the contents of which is incorporated herein in its entirety.

### GOVERNMENT FUNDING

This invention was made with government support under Grant Nos. P50CA150964, U01CA152756, U54CA163060 awarded by The National Institutes of Health. The United States government has certain rights to the invention.

### FIELD OF THE INVENTION

The present disclosure relates to a device suitable for collecting biological samples. In particular, the present disclosure relates to a device designed to specifically capture samples within the target areas of a patient for specific cell collection for particular diagnosis.

### BACKGROUND

Generally, Graft Versus Host Disease (GVHD), Cytomegalovirus Esophagitis (CMV), Candidiasis, and other infectious or inflammatory diseases can be diagnosed by pathological examination of endoscopic biopsies. GVHD most commonly occurs in the background of bone marrow transplantation. CMV can be diagnosed by the identification of cells with morphologic features of Cytomegalovirus (CMV Inclusions) on a pathological specimen. CMV infections generally occur in immunocompromised patients. Pathological specimens can be used for this diagnosis can be obtained from a tissue biopsy; or from a cytology specimen. Both of these types of samples are usually obtained via an endoscopic procedure. Candida infection can be diagnosed by the identification of fungal organisms with morphologic features of Candida on a pathological specimen. Candida infections generally occur in immunocompromised patients.

### SUMMARY

There is a need for improvements for collecting biological samples. The present disclosure is directed toward further solutions to address this need, in addition to having other desirable characteristics. Specifically, the present disclosure relates to collection of biological samples for diagnostic purposes for target diseases including Graft Versus Host Disease (GVHD), Cytomegalovirus Esophagitis (CMV), Candidiasis, and infectious diseases (e.g., Chagas Disease).

In accordance with example embodiments of the present disclosure, a device for collecting a sample including cells in a patient is provided. The device includes a collection portion attached to a tubular member, the collection portion and the tubular member provided with a size and shape sufficiently dimensioned to be swallowed by a patient. The collection portion having a first axial end portion and a second axial end portion designed to be actuated from a collapsed position and an expanded position for collecting the sample, the second axial end portion extending axially into the first axial end portion and having a concave shape when in the collapsed position.

In accordance with aspects of the present disclosure, the second axial end portion can have an outer surface facing radially outwardly when the second axial end portion is in the expanded position, the outer surface facing radially inwardly when the second axial end portion is in the collapsed position. The second axial end portion can include a plurality of tissue collecting projections for collecting the sample including the targeted cells. At least one of the tissue collecting projections can have a V-shape. The at least one of the tissue collecting projections can have first and second sides extending from an intersection at an angle to each other, the first and second sides extending toward the first axial end portion from the intersection when the second axial end portion is in the expanded position. Circumferentially extending ribs can extend between adjacent tissue collecting projections. The plurality of tissue collecting projections can include at least one circumferentially extending rib. The plurality of tissue collecting projections can include at least one radially extending cylindrical projection. The plurality of tissue collecting projections can include at least one bi-directional curved shaped projection. The plurality of tissue collecting projections can include at least one X-shaped projection.

In accordance with aspects of the present disclosure, the first and second axial end portions can be integrally formed as one-piece. The first and second axial end portions can be connected by a circumferentially extending hinge. The second axial end portion can move axially relative to the first axial end portion as the second axial end portion moves between the collapsed and expanded positions. The second axial end portion can expand to a sufficiently large size to capture the sample including the eosinophil white blood cells.

In accordance with example embodiments of the present disclosure, a method for collecting a sample including the targeted cells in a patient is provided. The method includes positioning a device provided with a collection portion having a first axial end portion and a second axial end portion at least one location within the body of the patient that is associated with at least one of GVHD, CMV, Candidiasis, Chagas Disease, axially moving the second axial end portion relative to the first axial end portion from a collapsed position within the first axial end portion into an expanded position, collecting the sample including the targeted cells with the second axial end portion in the expanded position, and axially moving the second axial end portion into the first axial end portion from the expanded position into the collapsed position to protect the collected cells. Depending on which of the diseases from a list of GVHD, CMV, Candidiasis, Chagas Disease being targeted, the device can be positioned at different locations, inflated to different sizes, and manipulated in different manners (e.g., rotated, push/pull, stationary, etc.) in order to specifically target the cells associated with the respective diseases.

In accordance with aspects of the present disclosure, the step of axially moving the second axial end portion from the collapsed position into the expanded position can include moving the second axial end portion from a concave shape to a convex shape. The step of axially moving the second axial end portion from the expanded position into the collapsed position can include moving the second axial end portion from a convex shape to a concave shape. The step of axially moving the second axial end portion from the expanded position into the collapsed position can include causing an outer surface of the second axial end portion facing radially outwardly when the second axial end portion is in the expanded condition to face radially inwardly when the second axial end portion is in the collapsed position. The step of moving the collection portion of the device into the body can include providing the device such that the collection portion can be inserted through the mouth to be swallowed, intubated, etc. The method can further include providing the second axial end portion with a plurality of tissue collecting projections designed to capture the sample including the targeted cells. The step of removing the device from within the body can include preventing the second end portion of the collection portion from engaging the esophagus at an area different from the collection site.

In accordance with aspects of the present disclosure, the step of axially moving the second axial end portion from the collapsed position into the expanded position can include applying pressurized air to the second axial end portion and the step of axially moving the second axial end portion from the expanded position into the collapsed position includes applying a vacuum to the collection portion. The method can further include performing at least one of pathological analysis, diagnostic analysis, and cell analysis on the sample including the targeted cells. The method can further include selecting the second axial end portion with a large diameter for adults and the second axial end portion with a small diameter for pediatrics or adolescents. The method can further include removing the device from the esophagus of the patient.

In one aspect, provided herein is a method for collecting a sample in a patient, the method comprising: positioning, at a location in the gastrointestinal tract associated with Graft Versus Host Disease (GVHD), a device provided with a collection portion having a first axial end portion and a second axial end portion; axially moving the second axial end portion, relative to the first axial end portion, from a collapsed position within the first axial end portion into an expanded position; and collecting the sample with the second axial end portion in the expanded position.

In another aspect, provided herein is a method for collecting a sample in a patient, the method comprising: positioning, at a location in an esophagus associated with Cytomegalovirus Esophagitis (CMV), a device provided with a collection portion having a first axial end portion and a second axial end portion; axially moving the second axial end portion, relative to the first axial end portion, from a collapsed position within the first axial end portion into an expanded position; and collecting the sample with the second axial end portion in the expanded position.

In another aspect, provided herein is a method for collecting a biological sample in a patient, the method comprising: positioning, at a location in at least one of mouth, throat, and vagina associated with Candidiasis, a device provided with a collection portion having a first axial end portion and a second axial end portion; axially moving the second axial end portion, relative to the first axial end portion, from a collapsed position within the first axial end portion into an expanded position; and collecting the sample with the second axial end portion in the expanded position.

In one embodiment of any of the aspects, the methods provided herein can further comprise axially moving the second axial end portion into the first axial end portion from the expanded position into the collapsed position to protect the collected cells.

In another embodiment of any of the aspects, the biological sample can include esophageal cells.

In another embodiment of any of the aspects, the step of axially moving the second axial end portion from the collapsed position into the expanded position can include moving the second axial end portion from a concave shape to a convex shape.

In another embodiment of any of the aspects, the step of axially moving the second axial end portion from the expanded position into the collapsed position can include moving the second axial end portion from a convex shape to a concave shape.

In another embodiment of any of the aspects, the step of axially moving the second axial end portion from the expanded position into the collapsed position can include causing an outer surface of the second axial end portion facing radially outwardly when the second axial end portion is in the expanded condition to face radially inwardly when the second axial end portion is in the collapsed position.

In another embodiment of any of the aspects, the step of moving the collection portion of the device into the gastrointestinal tract can include swallowing the collection portion.

In another embodiment of any of the aspects, the methods provided herein can further comprise providing the second axial end portion with a plurality of tissue collecting projections designed to capture the sample including esophageal cells.

In another embodiment of any of the aspects, the step of removing the device from the gastrointestinal tract can include preventing the second end portion of the collection portion from engaging the gastrointestinal tract at an area different from the collection site.

In another embodiment of any of the aspects, the step of axially moving the second axial end portion from the collapsed position into the expanded position can include applying pressurized air to the second axial end portion and the step of axially moving the second axial end portion from the expanded position into the collapsed position includes applying a vacuum to the collection portion.

In another embodiment of any of the aspects, the methods provided herein can further include performing at least one of pathological analysis, diagnostic analysis, and cell analysis on the sample including esophageal cells.

In another embodiment of any of the aspects, the methods provided herein can further include detecting at least one morphological, histological, or biological marker of GVHD in the sample including esophageal cells.

In another embodiment of any of the aspects, the presence of at least one morphological, histological, or biological marker of GVHD can indicate that the patient has or is suspected of having GVHD.

In another embodiment of any of the aspects, the methods provided herein can further include detecting at least one morphological, histological, or biological marker of CMV in the sample including esophageal cells.

In another embodiment of any of the aspects, the presence of at least one morphological, histological, or biological marker of CMV can indicate that the patient has or is suspected of having CMV.

In another embodiment of any of the aspects, the methods provided herein can further include removing the device from the gastrointestinal tract of the patient.

In another embodiment of any of the aspects, the sample can include buccal cells, squamous cells, vaginal cells, and/or Candida cells.

In another embodiment of any of the aspects, the step of moving the collection portion of the device into the at least one of mouth, throat, and vagina can include swallowing the collection portion.

In another embodiment of any of the aspects, the methods provided herein can further comprise providing the second axial end portion with a plurality of tissue collecting projections designed to capture the sample including buccal cells, squamous cells, vaginal cells, and/or Candida cells.

In another embodiment of any of the aspects, the step of removing the device from the at least one of mouth, throat, and vagina can include preventing the second end portion of the collection portion from engaging the at least one of mouth, throat, and vagina at an area different from the collection site.

In another embodiment of any of the aspects, the methods provided herein can further include performing at least one of pathological analysis, diagnostic analysis, and cell analysis on the sample including buccal cells, squamous cells, vaginal cells, and/or Candida cells.

In another embodiment of any of the aspects, the methods provided herein can further comprise detecting at least one morphological, histological, or biological marker from Candidiasis from the sample including buccal cells, squamous cells, vaginal cells, and/or Candida cells.

In another embodiment of any of the aspects, the presence of at least one morphological, histological, or biological marker of Candidiasis from the sample including buccal cells, squamous cells, vaginal cells, and/or Candida cells can indicate that the patient has or is suspected of having Candidiasis.

In another embodiment of any of the aspects, the methods provided herein can further include removing the device from the at least one of the mouth, throat, and vagina of the patient.

### BRIEF DESCRIPTION OF THE FIGURES

These and other characteristics of the present disclosure will be more fully understood by reference to the following detailed description in conjunction with the attached drawings, in which:

FIG. 1 is a schematic pictorial view of a biological sample collection device constructed in accordance with the present disclosure;

FIG. 2 is a schematic pictorial view of the collection device of FIG. 1 shown in a collapsed position;

FIG. 3 is a sectional view of the collection device of FIG. 2;

FIG. 4 is an enlarged plan view of a projection or bristle of the collection device of FIG. 1;

FIG. 5 is a sectional view of the projection taken along the line 5-5 in FIG. 4; and

FIG. 6 is a schematic pictorial view of the collection device showing a stylet and connector of the collection device.

### DETAILED DESCRIPTION

An illustrative embodiment of the present disclosure relates to device suitable for collecting biological samples for diagnostic purposes. The device can be designed to collect different types and quantities of biological samples using a combination of expandable textured surfaces. In some embodiments, the sampling device can be the device discussed with respect to U.S. application Ser. No. 15/103,638 and U.S. application Ser. No. 16/610,115, 63/030,547 all incorporated by reference in their entirety. The device of the present disclosure can also be designed to target specific types of biological samples for particular types of medical conditions and/or subsequent diagnosis. For example, the device can be used to target cell collection for diagnosis of GVHD, CMV, Candidiasis, Chagas Disease, or other diseases.

Graft Versus Host Disease (GVHD) commonly occurs in transplant patients when white blood cells of the donor's immune system remain within the donated tissue and target the body of the recipient host as being foreign and attacking that system. GVHD can occur within different parts of the body, such as for example, in the esophageal and/or gastrointestinal tract. When GVHD occurs in the GI tract it can be diagnosed by a biopsy of cells to measure for the presence of epithelial degeneration and apoptotic bodies within the collected cells. GVHD samples can also be collected by the device to facilitate this diagnosis whether by cytological/pathological examination, including cell blocks, or for molecular analysis. The device and system of the present disclosure can be used to position a collection surface within the esophageal and/or gastrointestinal tract and can be used to safely collect and remove the esophageal and/or gastrointestinal tract cells for diagnostic purposes. To diagnose GVHD, the device will be inflated after it has passed into the stomach and remain dilated throughout the course of the esophagus. When present in the proximal esophagus the device will be deflated and removed from the patient.

Cytomegalovirus Esophagitis (CMV) is a form of esophagitis caused by the cytomegalovirus. CMV can be diagnosed by a biopsy of cells within the esophagus to collected cells (or portions of lesions) for histological evaluation to identify enlarged cells in the epithelial and sub-epithelial layer containing the appropriate inclusion bodies. For CMV, other diagnostic methods can be used, such as for example, immunohistochemical staining. The device and system of the present disclosure can be used to position a collection surface within the esophagus and can be used to safely collect and remove the esophageal cells for diagnostic purposes, whether by cytological examination, including cell blocks, or for molecular analysis. To diagnose CMV, the device can be inflated after it has passed into the stomach and remain dilated throughout the course of being pulled through the esophagus. When located in the proximal esophagus the device can be deflated and removed from the patient.

Candidiasis or thrush is a fungal infection that can affect different areas of the body, such as the mouth, throat, vagina, etc. To diagnose Candidiasis, a sample of the infected area can be collected to be analyzed for the presence of organisms causing the infection. For example, for diagnosis of a yeast infection (e.g., within the vagina), a sample can be combined with potassium hydroxide (KOH) to isolate and identify Candida cells causing the infection. The device and system of the present disclosure can be used to position a collection surface within the respiratory, gastrointestinal, or esophageal systems and can be used to safely collect and remove the buccal, squamous, vaginal, and/or Candida cells for diagnostic purposes, whether by cytological examination, including cell blocks, including use of special stains—such as Grocott's Methenamine Silver (GMS) stain or for molecular analysis. To diagnose Candidiasis, the device can be inflated after it has passed into the stomach and remain dilated throughout being pulled through the esophagus. When present in the proximal esophagus the device can be deflated and removed from the patient.

Chagas Disease is a parasitic disease caused by Trypanosoma cruzi (or T. cruzi) and is commonly spread by insects. To diagnose Chagas Disease, a blood sample can be obtained to be analyzed for the presence of T. cruzi. The device and system of the present disclosure can be used to position a collection surface within a location where T. cruzi are frequently and can be used to safely collect and remove the cells for diagnostic purposes.

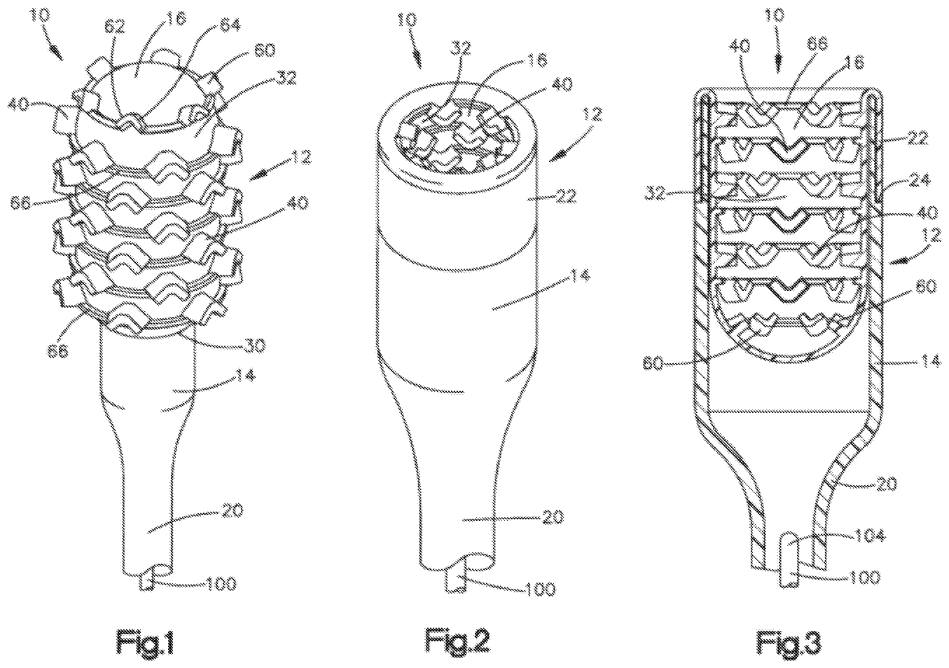

FIGS. 1 through 6, wherein like parts are designated by like reference numerals throughout, illustrate an example embodiment or embodiments of improved operation for biological cell collection, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIGS. 1-3, in some embodiments, sampling of cells or other substances can be performed using an example collection device 10. The collection device 10 can include a generally hollow longitudinally extending collection portion 12. The collection portion 12 can have a first or proximal axial end portion 14 connected to a second or distal axial end portion 16. The distal end portion 16 can have a first axial end portion 22 connected to the proximal axial end portion 14. The first end portion 22 may be connected to the proximal end portion 14 in any desired manner, such as by using an adhesive, welding, mechanical connection, bonding, or a combination thereof. In some embodiments, the first axial end portion 22 can engage a shoulder 24 on the proximal axial end portion 14. Therefore, the collection portion 12 can have a smooth outer surface. The distal axial end portion 16 may be connected to the proximal end portion 14 in any desired manner. The proximal axial end portion 14 and the distal axial end portion 16 may be made of a flexible polymer, such as silicone or polyurethane. In some embodiments, the distal axial end portion 16 can have a lower durometer than the proximal axial end portion 14. The distal axial end portion 16 may have a durometer between 5-90 Shore A. The durometer of the distal axial end portion 16 is preferably between 20-70 Shore A, and more specifically, approximately 30 Shore A. Any combination of elements and/or characteristics of the collection device 10 can be modified and/or specifically designed for capturing target cells/substances for any of the above-noted diseases.

In some embodiments, the distal axial end portion 16 may expand and contract. In some embodiments, the first or proximal axial end portion 14 is relatively rigid. Therefore, the proximal end portion 14 can have a fixed radial extent. The first axial end portion 14 and the second axial end portion 16 may be formed as separate pieces that are connected together in any desired manner or may be integrally formed as one-piece. Although the proximal end portion 14 is illustrated as having a cylindrical shape, the proximal end portion may have any desired shape.

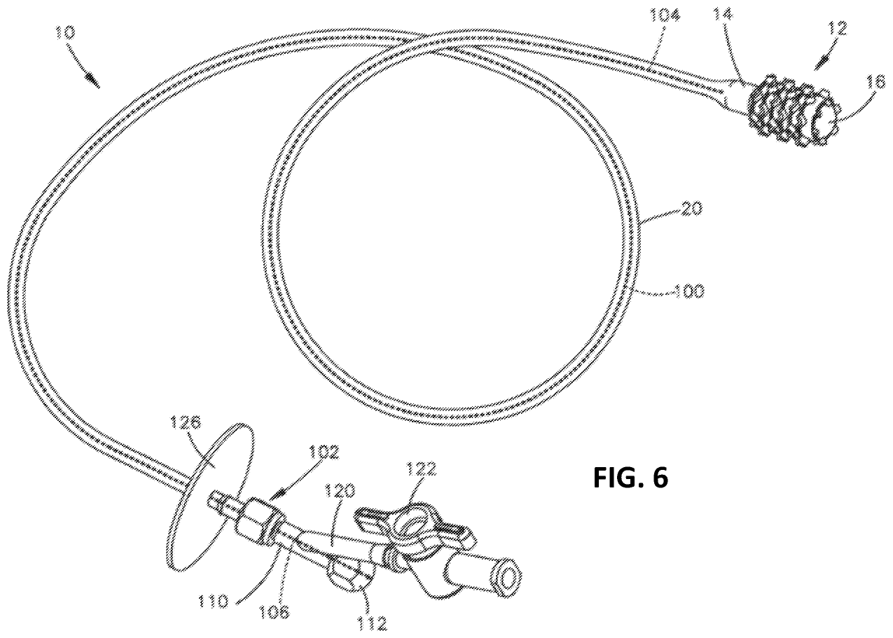

In some embodiments, the proximal axial end portion 14 can be connected to a support member 20, such as a tubular member or catheter, as discussed in greater detail with respect to FIG. 6. The support member 20 may be a tubular member in fluid communication with the interior of the collection portion 12. The proximal axial end portion 14 can conduct fluid, such as air, from the support member 20 to the distal axial end portion 16. In some embodiments, the support 20 can resist collapsing when a vacuum can be applied to the support member and resists stretching during withdrawal of the collection device 10 from the collection site.

In some embodiments, the second or distal end portion 16 of the collection portion 12 can have an expanded or inflated position (as shown in FIG. 1) and a collapsed or deflated position (as shown in FIGS. 2-3). The expanded position shown in FIG. 1 may be one of many expanded positions for the distal end portion 16. It is contemplated that the distal end portion 16 may expand more than shown in FIG. 1 so that the distal end portion obtains a more spherical shape and looks similar to a hot air balloon. In some embodiments, the distal end portion 16 can have a convex shape, shown in FIG. 1, when in the expanded or inflated position. The distal end portion 16 may extend radially outward a greater distance than the proximal end portion 14 when in the expanded position.

In some embodiments, the distal axial end portion 16 can be modified for a particular biological sample collection and/or collection at a particular location. For example, the distal axial end portion 16 can be specifically designed to capture the cells and/or substances needed to diagnose any of the GVHD, CMV, Candidiasis, Chagas Disease or other diseases. In some embodiments, the distal axial end portion 16 can be designed to expand more in a longitudinal direction for contacting sidewalls for collecting samples including white eosinophil blood cells at the esophagus, stomach, and the small intestine. For example, the distal axial end portion 16 can expand in in a longitudinal direction that is greater in length than the proximal end portion 14. The greater length of the distal axial end portion 16 can be preferred for contacting surfaces for capturing an adequate amount of sample that is sufficient to accurately count target cells within a sample for diagnosing different diseases.

In some embodiments, the distal axial end portion 16 can be sized and dimensioned for collecting a sample at a particular target location and/or subject. For example, the distal axial end portion 16 can be designed to expand to a larger diameter for an adult and a small diameter for a pediatric or adolescent. Similarly, different diameter sizes for the distal axial end portion 16 can be used for collecting samples at different locations, for example, collecting samples within the stomach may use a different diameter distal axial end portion 16 than when collecting samples within the esophagus. In another example, the distal axial end portion 16 can have a particular length dimension and shape for patients of different ages, weight, height, etc. Additionally, the distal axial end portion 16 can be expanded to different levels of pressure/stiffness to target particular samples to be collected.

In some embodiments, the distal end portion 16 can extend into the first or proximal axial end portion 14 and can have a concave shape, shown in FIGS. 2 and 3, when in the collapsed or deflated position. The distal end portion 16 may be inverted when in the collapsed position. The distal end portion 16 can extend axially into the interior of the proximal end portion 14 when in the collapsed or deflated position. Therefore, the distal end portion 16 can move axially or longitudinally relative to the proximal end portion 14 when moving between the deflated and inflated positions. In some embodiments, a relatively lower durometer of the distal end portion 16 allows the distal end portion to extend axially into the interior of the proximal end portion 14 and have a concave shape when in the collapsed position. The distal end portion 16 may be biased into the collapsed or deflated position in any desired manner.

In some embodiments, the proximal end portion 14 can have a relatively high durometer so that the proximal end portion does not collapse when a vacuum is applied to the proximal end portion through the support 20. The shape of the proximal end portion 14 does not change when the distal end portion 16 moves between the deflated and inflated positions. The proximal end portion 14 does not move radially when the distal end portion 16 moves between the deflated and inflated positions.

In some embodiments, the durometer or the proximal end portion 14 and the distal end portion 16 can be modified for collection of a particular biological sample type and/or collection at a particular location. For example, the proximal end portion 14 and the distal end portion 16 can be modified to traverse and/or operate differently when sampling in the esophagus compare to the GI tract. In some embodiments, the durometer and/or the proximal end portion 14 and the distal end portion 16 can be specifically selected for collecting samples located at different locations, such as for example the esophagus, stomach, and the small intestine. For example, the distal end portion 16 can have a sufficient durometer level to allow the distal end portion 16 to sufficiently maintain its expanded shape during sampling. In other words, the durometer of the distal end portion 16 can have a sufficiently high durometer to prevent the distal end portion 16 from collapsing when interacting with a tissue wall of the patient such that projections or bristles 40 thereon can collect an adequate level of sample material to test for any of GVHD, CMV, Candidiasis, Chagas Disease or other diseases. Although the example discusses the use of a high level of durometer, depending on the shape of the distal end portion 16, the projections or bristles 40 thereon, and the sampling location the durometer level can be higher or lower to most efficiently collect an adequate level of sample material having the target sample material for diagnosing particular diseases or conditions.

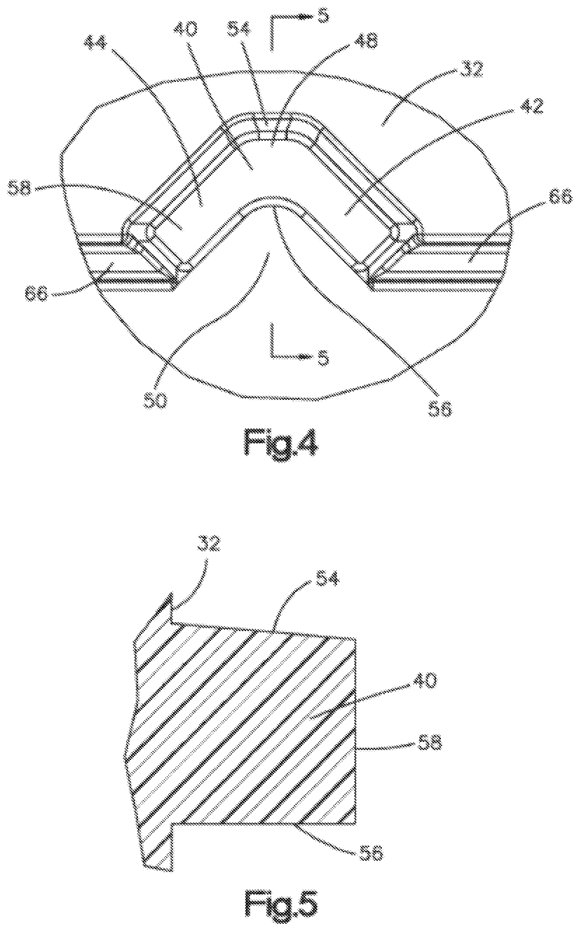

Referring to FIGS. 4 and 5, in some embodiments, the distal end portion 16 can have an outer surface 32 for collecting tissue when the distal portion is in the expanded position. The outer surface 32 can face radially outwardly when the distal end portion 16 is in the expanded position and may face radially inwardly when the distal end portion is in the collapsed or inverted position. It is contemplated that the outer surface 32 of the distal end portion 16 may have any desired construction for collecting tissue. In some embodiments, the outer surface 32 of the distal end portion 16 may have a plurality of projections or bristles 40 for collecting tissue. The distal end portion 16 may have any desired number of projections or bristles 40.

In some embodiments, the projections or bristles 40 may have a V-shape (FIG. 4). Each projection 40 can have a first side 42 and a second side 44 extending from an intersection 48. The first and second sides 42, 44 extend in a generally proximal direction from the intersection 48 when the distal end portion 16 is in the expanded position (as shown in FIG. 1). The first and second sides 42, 44 can extend in a generally distal direction when the distal end portion 16 is in the collapsed or inverted position (as shown in FIGS. 2 and 3). In some embodiments, the first and second sides 42, 44 can define a cup 50 for receiving collected biological samples. The cup 50 faces in a proximal direction when the distal portion 16 is in the expanded position and can face in a distal direction when the distal portion is in the collapsed position.

In some embodiments, the first and second sides 42, 44 may extend at an angle of approximately 90° relative to each other. It is contemplated that the first and second sides 42 and 44 may extend at any desired angle relative to each other. The desired angle may be determined based on the type of biological sample to be collected. Alternatively, the projections 40 may be cup shaped or have a semi-circular shape.

In some embodiments, each of the projections or bristles 40 can have side walls 54 and 56 (as shown in FIG. 5) that extend radially outward from the outer surface 32 when the distal portion 16 is in the expanded position. The side wall 56 faces the proximal direction when the distal portion is in the expanded position and forms an inner side of the cup 50. The side wall 54 faces the distal direction when the distal portion is in the expanded position and forms an outer wall of the cup 50. The side walls 54 and 56 extend from the outer surface 32 to a radially outer surface 58 of the projection 40. The side wall 56 extends generally perpendicular to the outer surface 32 and the outer surface 58 of the projection 40 when the distal end portion 16 is in a non-inflated position between the expanded and collapsed positions. The side wall 54 can taper toward the side wall 56 as the side wall 54 can extend from the outer surface 32 toward the radially outer surface 58 of the projection 40 when the distal end portion 16 is in the non-inflated position.

In some embodiments, the distal end portion 16 may include a plurality of projections or bristles 60 (as shown in FIG. 1) extending from a distal portion of the distal end portion 16. The projections 60 can have the same general V-shape as the projections 40 and can be smaller than the projections 40. In some embodiments, the projections 60 can have first and second sides 62 and 64 that have a length smaller than the first and second sides 42, 44 of the projections 40.

In some embodiments, the projections or bristles 40, 60 can be arranged in circumferentially extending rows (as shown in FIG. 1). It is contemplated that each row can have six projections 40 or 60. It is contemplated that each of the rows may have any desired number of projections 40 or 60. Each of the projections 40, 60 can be circumferentially offset from the projections on an adjacent row. Ribs 66 can extend circumferentially between adjacent projections 40, 60 in each row. The ribs 66 can extend between ends of the side walls 54, 56 opposite the intersections 48.

In some embodiments, the projections or bristles 40, 60 can be specifically sized, shaped, constructed from a material, etc. tailored for capturing a particular type of biological sample. For example, the projections or bristles 40, 60 can be designed with larger protrusions and/or with a higher level of durometer for capturing targeted cells at least one of the esophagus, stomach, the small intestine, large intestine, etc.

Similarly, the projections or bristles 40, 60 can be designed with different shapes for specifically capturing targeted cells at the esophagus, stomach, small intestine, large intestine, etc. In some embodiments, the projections or bristles 40, 60 can be designed to collect biological samples using a scraping process or a swabbing process. For example, the projections or bristles 40, 60 can have a coarse and/or be constructed from a hard material to obtain a larger, deeper, or targeted biological sample.

Referring to FIG. 6, in some embodiments, the catheter 20 may have a stylet 100 that provides stiffness to the catheter 20 so that a physician or operator may place the collection portion 12 into the back of a throat of a patient for easier swallowing.

The stylet 100 may extend through the catheter 20 from adjacent the first or proximal axial end portion 14 of the collection portion 12 to a connector 102. In some embodiments, the connector 102 can be connected with the catheter 20 and permits the introduction of fluid into the catheter for expanding the distal end portion 16 of the collection portion 12. In some embodiments, the stylet 100 can be preferably made of a polyether ether ketone (PEEK) polymer. However, the stylet may be a stainless-steel guidewire, a polymer monofilament extrusion and/or a stainless-steel monofilament core wire. The stylet 100 may have a rounded flexible distal end 104 (as shown in FIG. 3) spaced from the collection portion 12. In some embodiments, the flexible distal end 104 may be a graduated ground tip for increased flexibility. The distal end 104 may be the most flexible portion of the stylet 100.

In some embodiments, a proximal end 106 (as shown in FIG. 6) of the catheter 20 can be connected to the connector 102. The connector 102 may be a Y-fitting with a first branch 110 connected to the proximal end 106 of the stylet 100. In some embodiments, the proximal end 106 of the stylet 100 can extend through the first branch 110 into a cap 112 that seals and closes the first branch. The proximal end 106 can be connected to the cap 112 and the first branch 110 with epoxy and cut off flush with the proximal end of cap 112. The epoxy may connect the cap 112 to the first branch 110. It is contemplated that the stylet 100 may be fixedly connected to the cap 112, such as by insert molding. The stylet 100 may then be inserted into the Y-fitting 102 and catheter 20 and connected to the Y-fitting by the cap. The stylet 100 could then be removed from the catheter 20 and Y-fitting 102 if desired. In some embodiments, the catheter 20 may be lubricated to permit removal of the stylet 100 from the catheter. It is also contemplated that the proximal end 106 may extend through a Tuohy-Borst adapter connected to the first branch 110 to allow a user to loosen the Tuohy-Borst adapter and remove the stylet 100 to reduce the stiffness of the catheter 20. It is also contemplated that the stylet may extend along the outside of the catheter 20.

In some embodiments, the Y-fitting 102 can have a second branch 120 extending at an angle to the first branch 110. The second branch 120 may have a stopcock 122 for opening and closing the second branch. A syringe may be connected to the second branch 120 for introducing a fluid, such as air, into the Y-fitting 102 and catheter 20 to expand the distal end portion 16 of the collection portion 12 and apply a vacuum to remove the fluid to collapse the distal end portion 16 after collecting a sample. The stopcock 122 may be used to retain the fluid in the catheter 20 and collection portion 12 when obtaining a sample. The stopcock 122 and syringe help to control the injection of fluid to move the distal end portion 16 between the collapsed and expanded positions.

In some embodiments, a disk 126 may be connected to a proximal end of the catheter 20 or the distal end of the connector 102. The disk 126 can extend radially away from the catheter 20 to prevent the connector 102 from being inserted into a patient's mouth and/or throat.

In operation, in some embodiments, the device 10 can be used to assist in the diagnosis of different diseases, such as GVHD, CMV, Candidiasis, Chagas Disease or other diseases. For diagnosing GVHD, CMV, Candidiasis, Chagas Disease or other diseases, the collection portion 12 can be moved to a collection site within a body lumen, such as an esophagus, tract, or an organ, with the distal end portion 16 in the collapsed or deflated position. In some embodiments, the collection portion 12 may be swallowed by a patient. The stylet 100 may be manipulated to place the collection portion 12 into the back of the throat of the patient to help with the swallowing of the collection portion. It is also contemplated that the patient may be intubated with the collection portion 12 attached to the catheter. The device can also be inserted into other body orifices.

In some embodiments, the distal end portion 16 may be held in the collapsed or deflated position, for example, by applying a vacuum to the collection portion 12 through the support 20. The support member 20 or catheter may have depth markings to determine the collection site within the patient's anatomy. The collection portion 12 may be moved to a location within the esophagus, stomach, the small intestine, and/or the large intestine and the distal end portion 16 of the collection portion 12 may be expanded when at the appropriate location. In some embodiments, to ensure that the distal end is at the right location any combination of steps can be used, such as using markers, using tactic feedback, fluoroscopic guidance, assisted imaging, etc. or a combination thereof. For example, for positioning within the esophagus, distal end portion 16 may be swallowed into the stomach, everted and expanded within the stomach, pulled against the esophagogastric junction (GEJ), and using a length marking on the catheter to determine the length of the esophagus to swab.

In some embodiments, the distal end portion 16 can be moved from the collapsed position to the expanded position when the collection portion 12 is at or near the collection site. For example, the syringe connected to the Y-fitting 102 may be activated to apply pressurized fluid, such as air, to the distal end portion 16 to cause the distal end portion to move axially from the collapsed position to the expanded position.

In some embodiments, the collection portion 12 can be moved or rotated within the esophagus or body lumen to collect a biological sample, such as, tissue, cells, protein, RNA, DNA, bodily fluids, or a combination thereof from the collection site when the distal end portion 16 is in the expanded position. The collection portion 12 can also be moved in specific locations to collect specific samples. For example, collecting samples including targeted cells at the esophagus, stomach, the small intestine, and/or large intestine. It is contemplated that the collection portion 12 is only moved in a proximal direction so that the expanded distal end portion 16 engages the collection site to collect biological samples. The depth markings on the support member 20 or catheter may be used as a guide in positioning the device at the desired location.

In some embodiments, any combination of GVHD, CMV and Candidiasis screening can be performed by examining cells derived from cells collected by the collection portion 12 traversing the entire esophagus. This may involve distension of the collection portion 12 as it passes throughout the entire length of the esophagus. The cells may be examined by cytology assessments, including use of cell blocks, by special stains such as GMS, immunohistochemistry or by molecular testing.

In some embodiments, the device 10 can be used to gather biological samples related to Graft Versus Host Disease (GVHD). For collecting biological samples for GVHD, the collection portion 12 can be advanced to a desired location within the esophageal and/or esophageal and/or gastrointestinal tract using the support member 20. Once at a sampling location within the esophageal and/or gastrointestinal tract, the distal end portion 16 can be expanded out from within the proximal axial end portion 14 and further expanded to contact a sidewall of the esophageal and/or gastrointestinal tract for sample collection. In some embodiments, the distal end portion 16 can include projections or bristles 40 specifically designed for collecting biological cells needed to diagnose for GVHD. For example, the projections or bristles 40 can be designed for the collection of cells containing bilirubin from the esophageal and/or gastrointestinal tract. When GVHD occurs in the GI tract it can be diagnosed by a biopsy of cells to measure for the presence of epithelial degeneration and apoptotic bodies within the collected cells. GVHD samples can also be collected by the device to facilitate this diagnosis whether by cytological/pathological examination, including cell blocks, or for molecular analysis. When GVHD occurs in the stomach/esophageal tract it can be diagnosed by inflating the distal end portion 16 of the device 10 after the collection portion 12 has passed into the stomach and remain dilated throughout the course of the esophagus. When present in the proximal esophagus the distal end portion 16 can be deflated and removed from the patient.

In some embodiments, the device 10 can be used to gather biological samples related to Cytomegalovirus Esophagitis (CMV). For collecting biological samples for CMV, the collection portion 12 can be advanced to a desired location within the esophagus using the support member 20. Once at a sampling location within the esophagus, the distal end portion 16 can be expanded out from within the proximal axial end portion 14 and further expanded to contact a sidewall of the esophagus for sample collection. In some embodiments, the distal end portion 16 can include projections or bristles 40 specifically designed for collecting biological cells needed to diagnose for CMV. For example, the projections or bristles 40 can be designed for the collection of esophageal cells to identify enlarged cells in the epithelial and sub-epithelial layer containing the appropriate inclusion bodies from the location within the body. To diagnose CMV, the distal end portion 16 of the device 10 can be inflated after it has passed into the stomach and remain dilated throughout the course of the esophagus. When present in the proximal esophagus the distal end portion 16 of the device 10 can be deflated and removed from the patient

In some embodiments, the device 10 can be used to gather biological samples related to Candidiasis. For collecting biological samples for Candidiasis, the collection portion 12 can be advanced to a desired location within the mouth, throat, or vagina using the support member 20. Once at a sampling location within the mouth, throat, or vagina, the distal end portion 16 can be expanded out from within the proximal axial end portion 14 and further expanded to contact a sidewall of the mouth, throat, or vagina for sample collection. In some embodiments, the distal end portion 16 can include projections or bristles 40 specifically designed for collecting biological cells needed to diagnose for Candidiasis. For example, the projections or bristles 40 can be designed for the collection of Candida cells from the location within the body. To diagnose Candidiasis, the distal end portion 16 of the device 10 can be inflated after it has passed into the stomach and remain dilated throughout the course of the esophagus. When present in the proximal esophagus the distal end portion 16 of the device 10 can be deflated and removed from the patient.

In some embodiments, the device 10 can be used to gather biological samples related to Chagas Disease. For collecting biological samples for Chagas Disease, the collection portion 12 can be advanced to a desired location within the respiratory tract, digestive tract, or other hollow organs using the support member 20. Once at a sampling location within the respiratory tract, digestive tract, or other hollow organ, the distal end portion 16 can be expanded out from within the proximal axial end portion 14 and further expanded to contact a sidewall of the respiratory tract, digestive tract, or other hollow organ for sample collection. In some embodiments, the distal end portion 16 can include projections or bristles 40 specifically designed for collecting biological cells needed to diagnose for Chagas Disease. For example, the projections or bristles 40 can be designed for the collection of blood cells from the location within the body.

After the biological sample is collected, the distal end portion 16 can be moved from the expanded position to the collapsed or inverted position. For example, the distal end portion 16 may be moved from the expanded position to the collapsed position by applying a vacuum to the collection portion 12 with the syringe connected to the Y-fitting 102. As the collection portion 12 moves out of the body lumen, the distal end portion 16 does not engage the body lumen and prevents the collected biological samples from being contaminated by tissue from areas along the body lumen different from the collection site. Once the collection device 10 is removed from the patient, the biological samples can be collected from the surface of the distal end portion 16. For example, the biological samples can be collected via a wash and/or the collection portion 12 or the distal end portion 16 may be cut from the support member 20 and deposited in a biological sample vial.

Once the distal end portion 16 has been removed from the support member 20, the sample can be sent for further analysis. In some embodiments, a collected sample can be preserved for transportation and/or storage of the sample at an analysis location. The sample can be preserved using any combination of methods. For example, the sample can be preserved by storage within a container (e.g., vial) in a preservative fluid (e.g., methanol, water, etc.), it can be cooled or frozen, placed within a slide/mounted, or a combination thereof. Once preserved, the samples can be sent to the analysis location.

The analysis on collected samples can include any combination of analysis. For example, the analysis can include any combination of pathological analysis, steward pathology, diagnostic assay, cell analysis, biometric assay, etc. The sample can be obtained for a specific type of diagnostic. For example, a pathologist can visually inspect the biological samples and count the number of target cells to determine if any of the GVHD, CMV, Candidiasis, Chagas Disease or other diseases is likely. The cells can be diagnosed using any combination of methods which can be designed for specific diseases or conditions.

For example, GVHD diagnosis can be performed by biopsy of cells to measure for the presence of epithelial degeneration and apoptotic bodies within the collected cells or by cytological/pathological examination, including cell blocks, or for molecular analysis. CMV diagnosis can be performed by evaluation to identify enlarged cells in the epithelial and sub-epithelial layer containing the appropriate inclusion bodies and/or immunohistochemical staining. Candidiasis diagnosis can be performed by cytological examination, including cell blocks, including use of special stains—such as Grocott's Methenamine Silver (GMS) stain or for molecular analysis. In some embodiments, the analysis can be performed using a combination of users and computer analysis. For example, a user can use specialized software or artificial intelligence systems to perform black box diagnostics, visual inspection, point of care diagnostics, or other analyses. For example, a computer implemented system can be used to visually inspect the biological samples to determine if they fall within a threshold indicative of any of GVHD, CMV, Candidiasis, or other diseases.

The distinguishing features, characterization, and/or diagnosis of a particular disease or disorder provided herein can be determined by the skilled practitioner following the collection of a sample by the methods and device provided herein.

Thus, in one aspect, provided herein is a method of collecting a sample from a subject that is at risk of having or a subject that is suspected of having a disease provided herein (e.g., GVHD, CMV, and Candidiasis). In another aspect, provided herein is a method of diagnosing a subject with a disease provided herein by the methods provided herein.

In another aspect, provided herein is a method of treating a subject with a disease provided herein, the method comprising: collecting a sample using the methods or device provided herein; characterizing the sample for morphological, histological, and/or biological features associated with a disease provided herein (e.g., according to Table 1); detecting at least one morphological, histological, or biological marker associated with a disease provided herein; and providing an appropriate treatment to the subject when at least one morphological, histological, or biological marker associated with a disease provided herein is present.

In a further aspect, provided herein is a method of treating a subject with a disease provided herein, the method can include: collecting a sample using the methods or device provided herein; determining if the sample has a morphological, histological or biological marker, e.g. according to Table 1, associated with a disease; and providing an appropriate treatment to the subject when at least one morphological, histological, or biological marker associated with a disease provided herein is present.

The markers of each disease are discussed further below and exemplary morphological, histological, and biomarkers are summarized in Table 1.

Graft Versus Host Disease (GVHD) can be characterized in a cell sample by cellular apoptosis and cell damage in the squamous mucosa with dyskeratotic keratinocytes associated with a lichenoid interface inflammatory cell infiltrate. Additional markers are discussed in detail, e.g., in Srinagesh H K, et al., “Biomarkers in acute graft-versus-host disease: new insights.” Ther Adv Hematol. 2019, the teaching of which are incorporated herein by reference in its entirety.

CMV can be characterized in a sample by cell size. For example, the enlargement of infected cells and enlargement of both the nucleus and cytoplasm. Large, single basophilic intranuclear inclusions are common in CMV as well as intracytoplasmic inclusions. The virus can also be detected via immunohistochemistry and/or PCR techniques known in the art.

Candida species can be recognized in samples by the presence of ovoid and/or elongated pseudohyphal yeast cells. Histological staining by Grocott-Gomori's (or Gömöri) methenamine silver stain (GMS) and Periodic acid-Schiff (PAS) staining can highlight Candida in tissue samples.

The diseases provided herein can be identified by the exemplary markers provided in Table 1 below.

TABLE 1
Exemplary Disease Markers

| | Morphological | | Biological Markers |
|---|---|---|---|
| Disease | Markers | Histological Markers | (Biomarkers) |
| Graft Versus Host | Apoptosis | Presence of | Presence of IL-2Rα, |
| Disease (GVHD) | Cell damage in | inflammatory cells, | TNFR-1, IL-8, and |
| | squamous mucosa | cell death, and damage | hepatocyte growth |
| | Dyskeratotic | (e.g., using an | factor (HGF); elevated |
| | keratinocytes | hematoxylin-eosin | levels of T-cell |
| | associated with a | stain) | immunoglobulin and |
| | lichenoid interface | | mucin domain-3 |
| | | | (TIM3), IL-6; ST2 and |
| | | | REG3α |
| Cytomegalovirus | Enlargement of | CMV staining in | Presence of CMV IgG; |
| Esophagitis (CMV) | infected cells, | CMV-infected cells | real-time PCR for |
| | Large single | (e.g., using an | detecting CMV DNA; |
| | basophilic | hematoxylin-eosin | and IL-6; |
| | intranuclear | stain) | |
| | inclusions | | |
| | Intracytoplasmic | | |
| | inclusions | | |
| Candidiasis | Ovoid and elongated | GMS and PAS staining | real-time PCR for |
| | pseudohyphal forms | of Candida in tissue | detecting Candidiasis; |
| | of Candida. | samples | presence of 1-3-β- |
| | | | DGlucan; Mannan- |
| | | | Anti-mannan; and |
| | | | Candida albicans germ |
| | | | tube antibody |
| | | | (CAGTA) |

Additional characteristics of the diseases and methods of identifying and diagnosing the diseases provided herein are further described, e.g., in Richard Demay et al., “The Art and Science of Cytopathology.” (1996), First Edition; Goldblum and Odze, “Surgical Pathology of the GI Tract, Liver, Biliary Tract and Pancreas.” Third Edition, 2015; Clancy C J, Nguyen M H. Diagnosing Invasive Candidiasis. J Clin Microbiol. 2018 Apr. 25; 56(5); and Dioverti M V, Razonable R R. “Cytomegalovirus. Microbiol Spectr.” 2016 August; 4(4), the teachings of each of which are incorporated herein by reference in their entireties.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

1. A method for collecting a biological sample in a patient, the method comprising:
positioning, at a collection site in a gastrointestinal tract associated with Graft Versus Host Disease (GVHD), a device provided with a collection portion having a first axial end portion and a second axial end portion;
axially moving the second axial end portion, relative to the first axial end portion, from a collapsed position within the first axial end portion into an expanded position; and
collecting the biological sample with the second axial end portion in the expanded position.

2. The method of claim 1, further comprising axially moving the second axial end portion into the first axial end portion from the expanded position into the collapsed position to protect the biological sample.

3. (canceled)

4. The method of claim 1, wherein the step of axially moving the second axial end portion from the collapsed position into the expanded position includes moving the second axial end portion from a concave shape to a convex shape, or wherein the step of axially moving the second axial end portion from the expanded position into the collapsed position includes moving the second axial end portion from a convex shape to a concave shape.

5. (canceled)

6. (canceled)

7. The method of claim 1, wherein the step of positioning the collection portion of the device into the gastrointestinal tract includes swallowing the collection portion.

8. (canceled)

9. (canceled)

10. (canceled)

11. The method of claim 1, further comprising performing at least one of pathological analysis, diagnostic analysis, and cell analysis on the biological sample including esophageal cells.

12. The method of claim 1, further comprising removing the device from the gastrointestinal tract of the patient.

13. The method of claim 1, further comprising detecting at least one morphological, histological, or biological marker of GVHD from the biological sample including esophageal cells, wherein the presence of at least one morphological, histological, or biological marker of GVHD indicates that the patient has or is suspected of having GVHD.

14. (canceled)

15. A method for collecting a biological sample in a patient, the method comprising:
positioning, at a collection site in an esophagus associated with Cytomegalovirus Esophagitis (CMV), a device provided with a collection portion having a first axial end portion and a second axial end portion;
axially moving the second axial end portion, relative to the first axial end portion, from a collapsed position within the first axial end portion into an expanded position; and
collecting the biological sample with the second axial end portion in the expanded position.

16. The method of claim 15, further comprising axially moving the second axial end portion into the first axial end portion from the expanded position into the collapsed position to protect the biological sample.

17. (canceled)

18. The method of claim 15, wherein the step of axially moving the second axial end portion from the collapsed position into the expanded position includes moving the second axial end portion from a concave shape to a convex shape, or wherein the step of axially moving the second axial end portion from the expanded position into the collapsed position includes moving the second axial end portion from a convex shape to a concave shape.

19. (canceled)

20. (canceled)

21. The method of claim 15, wherein the step of positioning the collection portion of the device into the esophagus includes swallowing the collection portion.

22. (canceled)

23. (canceled)

24. (canceled)

25. The method of claim 15, further comprising performing at least one of pathological analysis, diagnostic analysis, and cell analysis on the biological sample including esophageal cells.

26. The method of claim 15, further comprising removing the device from the esophagus of the patient.

27. The method of claim 15, further comprising detecting at least one morphological, histological, or biological marker of CMV from the biological sample including esophageal cells, wherein the presence of at least one morphological, histological, or biological marker of CMV indicates that the patient has or is suspected of having CMV.

28. (canceled)

29. A method for collecting a biological sample in a patient, the method comprising:
positioning, at a collection site in at least one of mouth, throat, and vagina associated with Candidiasis, a device provided with a collection portion having a first axial end portion and a second axial end portion;
axially moving the second axial end portion, relative to the first axial end portion, from a collapsed position within the first axial end portion into an expanded position; and
collecting the biological sample with the second axial end portion in the expanded position.

30. The method of claim 29, further comprising axially moving the second axial end portion into the first axial end portion from the expanded position into the collapsed position to protect the biological sample.

31. The method of claim 29, wherein the biological sample includes buccal cells, squamous cells, vaginal cells, and/or Candida cells.

32. The method of claim 29, wherein the step of axially moving the second axial end portion from the collapsed position into the expanded position includes moving the second axial end portion from a concave shape to a convex shape, or wherein the step of axially moving the second axial end portion from the expanded position into the collapsed position includes moving the second axial end portion from a convex shape to a concave shape.

33. (canceled)

34. (canceled)

35. The method of claim 29, wherein the step of positioning the collection portion of the device into the at least one of mouth, throat, and vagina includes swallowing the collection portion.

36. (canceled)

37. (canceled)

38. (canceled)

39. The method of claim 29, further comprising performing at least one of pathological analysis, diagnostic analysis, and cell analysis on the biological sample including Candida cells.

40. The method of claim 29, further comprising removing the device from the at least one of mouth, throat, and vagina of the patient.

41. The method of claim 29, further comprising detecting at least one morphological, histological, or biological marker of Candidiasis from the biological sample including buccal cells, squamous cells, vaginal cells, and/or Candida cells, wherein the presence of at least one morphological, histological, or biological marker of Candidiasis from the biological sample including buccal cells, squamous cells, vaginal cells, and/or Candida cells indicates that the patient has or is suspected of having Candidiasis.

42. (canceled)
